(19) United States
(12) Patent Application Publication
(10) Pub. No.: US 2023/0218496 A1
(43) Pub. Date: Jul. 13, 2023

(54) IONIC LIQUID AS AN ANTIPERSPIRANT

(71) Applicant: Novigo GmbH, Grünberg (DE)

(72) Inventors: Thomas Schmidts, Giessen (DE); Michael Merzhäuser, Wetzlar (DE); Frank Runkel, Buseck-Oppenrod (DE)

(21) Appl. No.: 17/997,499

(22) PCT Filed: Apr. 28, 2021
(86) PCT No.: PCT/EP2021/061206

§ 371 Date: Oct. 28, 2022

(30) Foreign Application Priority Data
Apr. 30, 2020 (DE) 10 2020 111 882.1

(51) Int. Cl. A61K 8/49; A61Q 15/00

(52) U.S. Cl. CPC A61K 8/4926; A61Q 15/00; A61K 2800/30

(57) ABSTRACT

The invention relates to an aluminum salt-free, antiperspirant, deodorizing, cosmetic and/or dermatological composition comprising at least one antiperspirant active ingredient, wherein the antiperspirant active ingredient is selected from the group of ionic liquids.

The invention relates to an aluminum salt-free antiperspirant, and in particular deodorizing, cosmetic and/or dermatological composition, according to the generic terms of claim 1 and to the use of an ionic liquid as an antiperspirant and/or antiperspirant active ingredient according to claim 7.

Cosmetic compositions for use against body odor and/or sweating comprise antiperspirant and/or deodorizing active ingredients.

Sweat is a watery secretion secreted by the human skin via sweat glands. Body odor occurs when sweat, which is odorless in itself, is decomposed by microorganisms such as staphylococci and corynebacteria.

Active ingredients to inhibit perspiration, so-called antiperspirants, are nowadays used almost exclusively in the form of aluminum salts. However, these active ingredients cause skin irritation and have been discredited for some years for causing or promoting breast cancer. Even though the studies on possible health hazards are inconsistent, there is great uncertainty among users and a desire for antiperspirant compositions without aluminum salts.

The effect of aluminum salts as antiperspirants against thermal sweating under normal physiological conditions has been very well studied, although the mechanism of action has not yet been clearly clarified.

Antiperspirants are one of the possible strategies for controlling body odor and are used, for example, in deodorants. In addition to influencing the activity of the sweat glands, as is the case with antiperspirants, there are also, for example, the deodorizing principles of odor masking and, in addition, control of the microorganisms involved in odor formation, especially by antimicrobial agents.

Antimicrobial agents are intended to prevent excessive growth of odor-causing bacteria without severely compromising the biological equilibrium of the skin's own flora which, for example, would allow pathogens to spread.

Just as in common usage, deodorant here is understood to mean only the cosmetic product against body odor, regardless of whether it contains an antiperspirant or not. Deodorants are variously known in the form of sticks, solutions, roll-ons, aerosols and pump sprays. These are essentially leave-on products.

Deodorizing active ingredients in deodorants are substances that mask the odor of sweat and/or act against odor-causing bacteria. They are to be distinguished from antiperspirants.

However, European legislation does not clearly distinguish between deodorizing and antiperspirant active ingredients. Only the efficacy of cosmetic products can be clearly demonstrated.

Legislation in the USA clearly distinguishes between deodorants or deodorizing active ingredients and antiperspirants. The guidelines of the Food and Drug Administration (FDA, USA health authority) stipulate that the term “antiperspirant” may only be used for those products that have been shown to reduce sweating by at least 20% in 50% of test subjects (FDA, 2003).

The object of the present invention is to eliminate the disadvantages of the prior art and to provide an alternative, aluminum salt-free composition for inhibiting perspiration and especially for reducing odors, which in particular has fewer side effects.

The main features of the invention are specified in the characterizing part of claim 1 and in claim 7. Configurations form the subject matter of claims 2 to 6 and 8 to 10.

The invention relates to an aluminum salt-free, antiperspirant, in particular additionally deodorizing and cosmetic and/or dermatological composition comprising at least one antiperspirant active ingredient, wherein the antiperspirant active ingredient is selected from the group of ionic liquids, the at least one antiperspirant active ingredient having at least one cation and at least one anion.

It has been found that ionic liquids based on a cation with a substituted basic structure of an: imidazolium, ammonium, phosphonium, guanidinium, pyrrolidinium, piperidinium and pyridinium, exhibit antiperspirant effects. In addition, they have an antimicrobial and deodorizing effect and show good skin compatibility. In addition, a harmful to health, or even mutagenic, teratogenic and/or carcinogenic effect can essentially be ruled out.

In addition, no permanent staining was observed when a product with the composition according to the invention was applied to textiles. This has an additional advantageous influence on a customer's purchase decision.

The composition can be used as a cosmetic product for normal sweating and is freely available commercially, for example in a drugstore and/or pharmacy. According to the European understanding, a dermatological composition, in contrast to a cosmetic composition, is a prescription or at least pharmacy-only product that is suitable for medical use, for example in the case of hyperhidrosis.

The composition is not limited to any particular product form and may be used, for example, in the form of sticks, solutions, roll-ons, creams, gels, aerosols, pump sprays and as impregnated flexible substrates, for example deodorant wipes.

The usability of the composition with antiperspirant active ingredient is not limited to a specific body area. In particular, application is possible on the underarms, on the feet and hands.

Ionic liquids are molten salts where the melting temperature is less than 100° C. Surprisingly, ionic liquids have been found to have an antiperspirant effect. In addition, ionic liquids have a deodorizing effect. At least in vitro, they have a pronounced efficacy against skin bacteria that are the cause of the sweat odor. Furthermore, they show a pronounced effect in terms of sweat reduction. As with aluminum chloride, there are currently various hypotheses regarding the exact mechanism of action.

Due to the antibacterial activity of the ionic liquids, a cosmetic and/or dermatological product having the composition according to the invention does not need additional preservatives. This not only saves costs in the manufacture of such a product, but it is also gentler on the body, since many preservatives exhibit irritation up to carcinogenic side effects.

In a preferred embodiment, the composition has a single substance from the group of ionic liquids as antiperspirant active ingredient, which in particular simultaneously exhibits a deodorizing effect. This allows the action of the substance to be employed in a targeted manner without affecting the body with further substances and, in the worst case, afflicting it.

Alternatively, it is provided that the composition comprises two or more substances from the group of ionic liquids, these acting together as antiperspirant active ingredients. As a result, effects that differ from one another can accumulate positively.

In a preferred development, the composition comprises further antiperspirant active ingredients in addition to one or more substances from the group of ionic liquids. As a result, further advantageous effects may be achieved in a product with the composition.

Ionic liquids have at least one cation and one anion.

The cation is selected from the group of: imidazolium, ammonium, phosphonium, guanidinium, pyrrolidinium, piperidinium and pyridinium, wherein the specified basic structures may in each case have one or more substituents, of which at least one substituent is an alkyl substituent having a chain length of 1 to 10 carbon atoms. The at least one alkyl substituent preferably has a chain length of 3 to 9 carbon atoms, particularly preferably 4 to 8 carbon atoms.

The anion is preferably selected from the group consisting of tetrafluoroborate, methyl sulfate, fluoride, chloride, bromide, iodide, tris(trifluoromethylsulfonyl)methide, trifluoromethanesulfonate, tosylate, bis(1,2-benzenediolato(2-))borates, bis(oxalato (2-))borates, 2-(2-methoxyethoxy)ethyl sulfate, thiocyanate, dicyanamide, octyl sulfate, methyl sulfonate, trifluoroacetate, hexafluorophosphate, ethyl sulfate, hydrogen sulfate, hexafluoroantimonate, bis(trifluoromethyl)imide, bis(pentafluoroethyl)phosphinate, bis(trifluoromethylsulfonyl)imides, tris(pentafluoroethyl)trifluorophosphates.

In a preferred embodiment, the at least one antiperspirant active ingredient has at least one cation and at least one anion, the cation being selected from the group of alkylpyridinium cations. The alkyl substituent has a chain length of Cl to 010 which corresponds to methyl to decane. The substituent can be branched or unbranched. The alkyl substituent is preferably positioned on the nitrogen atom of the pyridine ring, in particular it takes the form of an N-alkylpyridinium cation. Preferably, the alkyl substituent of the pyridinium cation has a chain length of 3 to 9 carbon atoms, particularly preferably 4 to 8 carbon atoms, especially preferably 5 to 7 carbon atoms.

In a preferred embodiment, the at least one antiperspirant active ingredient has at least one cation and at least one anion, the anion being selected from the group of halides. The group of halides comprises in particular fluoride, chloride, bromide and iodide.

The at least one antiperspirant active ingredient preferably comprises 1-hexylpyridinium chloride. Surprisingly, this ionic liquid was found to have a very good antiperspirant, antimicrobial and deodorizing effect with good skin compatibility. In addition, a harmful to health, or even mutagenic, teratogenic and/or carcinogenic effect can essentially be ruled out.

In addition, the use of 1-hexylpyridinium chloride when applied directly to white or black textiles does not result in any permanent stains after washing. It is therefore a stain-free alternative to commercially available antiperspirants in deodorants, which often result in the formation of stains on textiles.

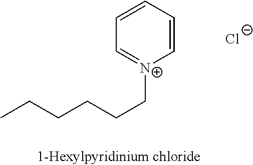

In a preferred embodiment, the at least one antiperspirant active ingredient is used in the composition to an extent of 0.5 to 30% by weight, particularly preferably 1 to 25% by weight, especially preferably 5 to 10% by weight, based on the total weight of the composition. A good antiperspirant effect of the ionic liquids, especially of 1-hexylpyridinium chloride, could be demonstrated at an active ingredient concentration of 0.5% by weight up to 30% by weight. In addition, at an active ingredient concentration of 0.5% by weight up to 25% by weight, serious side effects were ruled out.

When used as a cosmetic and/or dermatological product, a concentration of 3 to 15% by weight of the antiperspirant active ingredient is particularly preferred in order to achieve a good effect with little or no side effects.

The composition preferably comprises the following ingredients to 100% by weight:
at least one antiperspirant active ingredient selected from the group of ionic liquids at a proportion of 0.5-30% by weight, preferably 1 to 25% by weight, particularly preferably 5-10% by weight,
70-99% by weight, preferably 90-95% by weight, water.

Such a composition, which only comprises an antiperspirant active ingredient from the group of ionic liquids and water, can be used, for example, as a pump spray.

In a further preferred embodiment, the composition comprises the following ingredients to 100% by weight:
at least one antiperspirant active ingredient selected from the group of ionic liquids at a proportion of 0.5-30% by weight, preferably 1-25% by weight, particularly preferably 5
10% by weight,
0-50% by weight, preferably 10-40% by weight, particularly preferably 20-30% by weight, ethanol,
0-5% by weight, preferably 1-3% by weight, of an antibacterial active ingredient
0-2.5% by weight, preferably 2-2.5% by weight, of at least one pH regulator
0-2.5% by weight, preferably 0.2-0.8% by weight of a chelating agent,
0-4% by weight, preferably 0.01-2% by weight, of at least one viscosity regulator,
0-2% by weight, preferably 0.2-1% by weight of at least one surfactant,
0-30.5% by weight, preferably 5.5-16.5% by weight, of at least one skin care substance,
0-5% by weight, preferably 1-2% by weight, of at least one fragrance,
0-99% by weight, preferably 10-75% by weight, particularly preferably 35-50% by weight, water,
0-99% by weight, preferably 10-80% by weight, particularly preferably 30-70% by weight, of propellant,
0-10% by weight, preferably 0.01-10% by weight, particularly preferably 0.1-7% by weight preservatives,
0-10% by weight, preferably 0.01-5.0% by weight antioxidant and
0-10% by weight, preferably 0.2-7% by weight, odor absorber.

Such a composition is suitable, for example, for use as a pump spray, as a spray, as a roll-on, as a cream and/or as a stick.

The antibacterial active ingredient used is, for example, ethylhexylglycerin, caprylyl glycol and/or glyceryl caprylate. In particular, the antibacterial active ingredient may also comprise an active ingredient that inhibits bacterial enzymes involved in the formation of odor-intensive substances, for example triethyl citrate.

The pH regulator comprises for example, lactic acid and/or salts thereof, citric acid and/or salts thereof. Hydrochloric acid and/or salts thereof and/or sodium hydroxide.

Sodium gluconate, sodium citrate, sodium EDTA (EDTA—ethylenediamine tetraacetate) and/or phytic acid are preferably used as chelating agents.

Viscosity regulators are, for example, carbomers, acrylic acids and/or derivatives, hyaluronic acid, cellulose and/or derivatives, in particular hydroxyethyl cellulose, natural thickeners, in particular gellan gum or carrageenan, xanthan and/or derivatives, polyethylene glycols and/or derivatives, fatty acids and/or -derivatives and/or waxes.

Preferred surfactants are anionic surfactants, particularly alkyl carboxylates and/or fatty alcohol sulfates, cationic surfactants, particularly stearalkonium chloride, non-ionic surfactants, particularly glyceryl stearate citrate and/or amphiphilic surfactants, particularly cocamidopropyl betaines.

Skin care ingredients can include a wide variety of substances and mixtures. Examples are natural and synthetic waxes, emollients and oils, particularly paraffin silicone oil, medium-chain triglycerides, octyldodecanol, avocado oil, almond oil, and D-panthenol, allantoin, ectoin, betaine, urea, vitamins and minerals, particularly magnesium and magnesium compounds and/or vitamin E and also prebiotic substances.

Water is preferably used in the form of distilled water.

Propane, n-butane and/or isobutane, for example, are used as propellant. A propellant is used especially in sprays.

If a preservative is used, this is preferably selected from the group of polyols, alcohols, parabens and/or active salts, such as salts of sorbic acid, benzoic acid and/or anisic acid.

An antioxidant is preferably selected from the group of: ascorbic acid, ascorbyl palmitate, butylhydroxytoluene, substituted hydroxyhydrocinnamic esters, substituted benzotriazoles, dialkylthiodialkanoates, tocopherol and esters thereof, in particular tocopheryl acetate, hydroxymethoxyphenyl decanone, paradol, substituted hydroxyhydrocinnamic esters and/or mixtures of these.

The odor absorbers that may be used are, for example, silicates, zeolites, zinc ricinoleate, cyclodextrins, metal oxides.

In a preferred embodiment, the composition has a pH of 3.5 to 7.0, particularly preferably pH 4 to 5.5. pH values below 3.5 are physiologically incompatible and lead to irritation and even chemical burns on the skin. The pH value of healthy skin is around 4-6.5, although it can be slightly higher in the underarms, for example, and is around pH 6.

The invention also relates to the use of an ionic liquid as antiperspirant and/or antiperspirant active ingredient. In particular, a substance or a mixture of substances selected from the group of ionic liquids is provided for use in a cosmetic and/or dermatological composition for sweat reduction as an antiperspirant.

In a preferred development, the ionic liquid, as antiperspirant active ingredient, additionally has a deodorizing effect. As a result, this not only inhibits perspiration, but also the development of odors caused by microorganisms.

The antiperspirant, in particular in a deodorant, is used directly on the skin, preferably in those areas that show increased perspiration, predominantly on the underarms, hands and feet.

For this application, the ionic liquid preferably comprises 1-hexylpyridinium chloride. 1-Hexylpyridinium chloride is thus useful as an antiperspirant. In cosmetic and/or dermatological products, this ionic liquid has a very good antiperspirant, sweat-inhibiting and deodorizing effect and is well tolerated. The active ingredient shows a pronounced effect against skin bacteria (in vitro) that are the cause of the odor of sweat. These include, in particular, representatives of Corynebacterium such as Staphylococcus epidermidis. Furthermore, it shows a pronounced effect in terms of sweat reduction (in vivo). In addition, the active ingredient 1-hexyl pyridinium chloride is well tolerated by the skin. In vitro evidence shows low toxicity, no skin corrosion, no mutagenicity or carcinogenicity. Good in vivo skin compatibility was also determined by way of example for 5% and 7.5% of the active ingredient.

Two theories on the mechanism of action of aluminum chloride are suspected: A) triggering inflammation causes the pore to close and backflow of the sweat. B) aluminum chloride precipitates proteins and clogs the pores. With regard to the mechanism of action of the antiperspirant active ingredients of the composition according to the invention, indications point to mechanism of action B).

A preferred embodiment provides for the ionic liquid to be used together with another antiperspirant active ingredient.

In an alternative embodiment of the use, the ionic liquid is used as the sole antiperspirant and in particular deodorizing active ingredient in a cosmetic and/or dermatological preparation or product.

The features related to the composition are transferable and to the use and vice versa. Further features, details and advantages of the invention result from the wording of the claims and from the following description of working examples.

### Example Formulations and Investigations of the Active Ingredient Properties

### 1. EXAMPLE FORMULATIONS

The % data in the formulations are to be understood as percentages by weight.

### Formulation Example 1—Roll-on (5% Active Ingredient)

| Phase | Ingredients | Proportion in % by weight |
|---|---|---|
| A | Ethanol | 40.0 |
| A | 3-(2-Ethylhexyloxy)propane-1,2-diol | 1.0 |
| A | Triethyl citrate | 4.0 |
| A | Perfume | 2.0 |
| B | Water | 43.5 |
| B | 75% D-Panthenol in water | 0.5 |
| B | 1-Hexylpyridinium chloride | 5.0 |
| C | Hydroxypropyl cellulose | 2.0 |
| C | Glycerin | 2.0 |

The ingredients of phase A and phase B are mixed within each phase respectively. Subsequently, phase A is mixed with phase B. The phase C ingredients are dispersed together. With each other. Phase AB, which corresponds to the mixture of phase A and phase B, is added to phase C and stirred until smooth. The overall formulation is adjusted to approximately pH 4.5 by adding citric acid.

### Formulation Example 2—Active Ingredient Solution 7.5%

| Phase | Ingredients | Proportion in % by weight |
|---|---|---|
| A | Water | 90.5 |
| A | 1-Hexylpyridinium chloride | 7.5 |
| A | Hydroxypropyl cellulose | 2.0 |

All ingredients of phase A are mixed together. The overall formulation is adjusted to approximately pH 4.5 by adding sodium hydroxide.

### Formulation Example 3—Active Ingredient Solution 10%

| Phase | Ingredients | Proportion in % by weight |
|---|---|---|
| A | Water | 88 |
| A | 1-Hexylpyridinium chloride | 10 |
| A | Hydroxypropyl cellulose | 2.0 |

All ingredients of phase A are mixed together. The overall formulation is adjusted to approximately pH 4.5 by adding sodium hydroxide.

### Formulation Example 4—Active Ingredient Solution 5%

| Phase | Ingredients | Proportion in % by weight |
|---|---|---|
| A | Water | 93 |
| A | 1-Hexylpyridinium chloride | 5 |
| A | Hydroxypropyl cellulose | 2.0 |

All ingredients of phase A are mixed together. The overall formulation is adjusted to approximately pH 4.5 by adding sodium hydroxide.

### 2. Investigation of the Active Ingredient Properties

It should be noted that the terms test product and test composition are used synonymously. The same applies to the terms composition and product, test person and test subject.

### 2.1 Investigation of Sweat Inhibition

### 2.1.1 Gravimetric Determination of the Antiperspirant Effect of a Test Product Having 5% Active Ingredient as a Roll-on (Example Formulation 1) (Short-Term Use)

The antiperspirant effect was investigated in a randomized, comparative and blinded manner on twelve test subjects, eight of whom were female and four male and all aged between 32 and 64. Three test subjects dropped out during the test.

To prepare for the test, during the so-called washout period, the test subjects were not allowed to apply any antiperspirant for 21 days. The application takes place over four consecutive days and once a day.

After applying 400-450 μL of the test product with a pipette and smearing it with a finger stall on a defined underarm, the test subjects were exposed twice for 20 minutes each time at a temperature of 38° C.±2° C. and a relative humidity of 35%±5%. The other underarm remained untreated throughout the test period.

Sweat production and sweat inhibition were measured prior to application, which corresponds to a base measurement point t0, and 24 h after the last application, which corresponds to a measurement point t24h. Sweat production was measured gravimetrically.

The direct comparison of the untreated reference measurement at the base measurement point t0 with a measurement 24 h after the last application of the product to be tested at measurement point t24h shows that the tested product causes a significant reduction in sweat.

The product tested has an average effectiveness of 87.3%. Even at low concentrations, the test product shows a relevant reduction in the amount of sweat of 13.7% after short-term use.

None of the test subjects showed a negative skin reaction during the course of the test.

### 2.1.2 Gravimetric Determination of the Antiperspirant Effect (Long-Term Use)

Further gravimetric investigations on sweat inhibition were performed as follows:

After a conditioning phase, in which the test subjects spent 17 days without deodorant having antiperspirant active ingredients and 5 days of those also without any deodorant, the test product was applied once a day to a defined underarm. The application period was four weeks. A reference test was analyzed after the conditioning phase and before the test product was applied for the first time, which is also referred to as the baseline sweat test, and approximately 16 hours after the last application.

The investigations were carried out randomly with respect to the test product, and a control product without an antiperspirant active ingredient, on the right and left underarms on a total of five test subjects, three female and two male aged 30 to 47 years. Only subjects who sweated at least 100 mg per underarm under the study conditions were included.

The sweat secreted from the underarms was determined gravimetrically in each case during intensive sporting activity, for example in the form of 30 minutes on a cross-trainer under defined conditions, without using the product to be tested as a reference study, and after a four-week application phase. For this purpose, cotton pads having a certain weight were attached beneath both underarms of the test subjects. The sweat absorbed by the cotton pads under stress conditions was determined gravimetrically.

### 2.1.2.1 Result of the Gravimetric Determination of the Antiperspirant Effect of a Test Product Having 5% Active Ingredient (Example Formulation 4)

Comparison of a baseline sweat test, which was carried out prior to the application period without using the test product, and a sweat test 16 hours after completion of the application of the test product resulted in a reduction in the amount of sweat in the test area in relation to the control area of 32% on average across the test subjects.

### 2.1.2.2 Result of the Gravimetric Determination of the Antiperspirant Effect of a Test Product Having 7.5% Active Ingredient (Example Formulation 2)

Comparison of a baseline sweat test, which was carried out prior to the application period without using the test product, and a sweat test 16 hours after completion of the application of the test product resulted in a reduction in the amount of sweat in the test area in relation to the control area of 39% on average across the test subjects.

### 2.2 Determination of Odor Intensity Via a Sniff Test

The odor intensity and the deodorizing effect of the test product that can be determined thereby were determined by means of a so-called sniff test as follows.

After a conditioning phase, in which the test subjects spent 17 days without deodorant having antiperspirant active ingredients and 5 days of those also without any deodorant, the test product with a fragrance-free formulation was applied once a day to a defined underarm. The application period was four weeks. A reference test, the so-called baseline sniff test, was analyzed after the conditioning phase and before the test product was applied for the first time, and approximately 16 hours after the last application.

A test product and a control product without antiperspirant active ingredient were applied daily, after randomization, under the right and left underarms for 28 days. A total of five test subjects, three female and two male aged between 30 and 47 years, were investigated.

The group of so-called sniffers consisted of five people, two of whom were female and three male, aged 40 to 48.

To check the effectiveness of the test product, a so-called baseline sniff test was initially conducted to determine the strength of the individual body odor of each test subject. For this purpose, each test subject had to wash both underarms with perfume-free soap and about 12 hours later specific non-scented cotton pads were attached beneath both underarms. These were worn for four hours in each case and individually sealed in airtight glass jars immediately after removal. These glass jars were labeled with numbers according to a blinding and randomization table and evaluated by to the sniffers with respect to odor intensity (baseline sniff test).

Approximately 16 hours after the last application of test product beneath an underarm, the odor intensity was determined again. Randomization and blinding were performed as described above. The sniffers independently evaluated the intensity of the sweat odor for both underarms.

### 2.2.1 Result of the Determination of the Odor Intensity of a Test Product Having 5% Active Ingredient (Example Formulation 4)

On average across the test subjects and sniffers, the baseline sniff test showed an odor intensity of level 2 beneath both underarms, which corresponds to a barely perceptible odor of sweat.

After the application period and 16 h after the last application of the fragrance-free test product, the odor intensity was determined again by means of the sniff test. On average across the test subjects and sniffers, there was a difference of one level of odor intensity between the odor intensity of the untreated underarm and that of the underarm previously treated with the test product. The odor intensity of the untreated underarms was evaluated at 2.2 on average, with 2 corresponding to a barely perceptible odor of sweat. The odor intensity of the underarms treated with test product was 0.9 on average, with 0 corresponding to no sweat odor.

### 2.2.2 Result of the Determination of the Odor Intensity of a Test Product Having 7.5% Active Ingredient (Example Formulation 2)

On average across the test subjects and sniffers, the baseline sniff test showed an odor intensity of level 2 beneath both underarms, which corresponds to a barely perceptible odor of sweat.

After the application period and 16 h after the last application of the fragrance-free test product, the odor intensity was determined again by means of the sniff test. On average across the test subjects and sniffers, there is a difference of one level of odor intensity between the odor intensity of the untreated underarm and that of the underarm treated with the test product. The odor intensity of the untreated underarms was evaluated at 1.8 on average, with 2 corresponding to a barely perceptible odor of sweat. The odor intensity of the underarms treated with test product was 1.0 on average, with 0 corresponding to no sweat odor.

### 2.3 Investigation of Skin Compatibility

The aim of examining skin compatibility, the so-called epicutaneous test, is to rule out primary skin irritation caused by a test product and/or existing sensitization to the test product.

The test substances are applied occlusively to the skin at suitable concentrations. The epicutaneous contact with the test product is limited in terms of location and time and is intensified by occlusion conditions, so that the absorption of the test substances is favored. The skin was examined after 24, 48 and 72 hours.

The occlusion conditions are used to allow a potential allergen to more easily penetrate through the stratum corneum to reach the effector cells that could elicit a local immune system response. A positive response occurs when a threshold level of irritation/sensitization is exceeded. A positive reaction to a correctly applied epicutaneous test is considered evidence of primary irritation by the test substance, but not necessarily evidence of sensitization.

For the test, 20 mg or 20 μL of the test product is applied undiluted to a ready-made plaster, which is applied and fixed to the clinically healthy skin of the upper back. The test plaster is removed after an exposure time of 24 hours and for the first time assessed dermatologically and allergologically. A second and third assessment was made after 48 and 72 hours. The test reaction was evaluated for the first time 30 minutes after removal of the test plaster under standardized lighting.

The test group consists of 30 adult, male and female test subjects, whereby test subjects with dry, oily, normal and sensitive skin were included within the test group.

### 2.3.1 Investigation of the Skin Compatibility of a Test Product Having 7.5% Active Ingredient (Formulation Example 2)

When examining the skin compatibility of a test product with the composition of formulation example 2, which corresponds to an active ingredient concentration of 7.5%, there were no skin changes in the test area in 29 of 30 test subjects after 24, 48 and 72 hours. The epicutaneous tests were carried out according to the international guidelines of the International Contact Dermatitis Research Group as described above.

One test subject developed a mild reaction after 24 and 48 hours, which was no longer visible after 72 hours.

### 2.4 Efficacy Against Odor-Causing Bacteria

Perspiration odor is mainly caused by the resident bacteria of the human microflora. Corynebacteria and germs of the genus Staphylococcus epidermidis are mainly found in the underarm area. Therefore, the bacteriostatic effect of a composition comprising 1-hexylpyridinium chloride in water was investigated in vitro on the relevant test microbes.

The test microbes used were Staphylococcus epidermidis with the identifier DSM 20044, ATCC 14990, Corynebacterium xerosis with the identification DSM 20743, ATCC 373 and Kocuria rhizophila of the type Micrococcus with the identifier DSM 11926. Using a series of dilutions of the substance to be tested, the minimum inhibitory concentration was determined at which growth of the bacteria in suspension could no longer be detected. 1-Hexylpyridinium chloride shows a bacteriostatic effect up to a concentration of 0.4% Staphylococcus epidermidis, in the case of Corynebacterium xerosis an inhibition of bacterial growth could even be observed up to a final preparation concentration of 0.2%. Thus, an antimicrobial effect on odor-forming microorganisms and thus a deodorizing effect of the active ingredient 1-hexylpyridinium chloride is also shown in vitro.

### 2.5 Investigation of Protein Precipitation In Vitro

The use of aluminum chloride beneath the underarm causes a reduction of perspiration. The most common mode of action of aluminum chloride discussed in the literature explains sweat reduction as clogging of the pores by precipitation of proteins by the aluminum chloride.

Therefore, the ability of a composition comprising 1-hexylpyridinium chloride in water for protein precipitation was investigated in vitro using BSA (bovine serum albumin) gelling experiments. An aqueous solution of aluminum chloride hexahydrate was used as a positive control.

It could be shown that the composition with 1-hexylpyridinium chloride also leads to gelation of BSA. It is therefore assumed that the effect of 1-hexylpyridinium chloride is analogous to the effect also discussed for aluminum chloride and that the precipitation of proteins leads to closure of the sweat pores.

### 2.6 Investigation of Staining on Textiles

Potential staining of a 7.5% by weight composition. 1-hexyl pyridinium chloride in water was investigated on white and black cotton textiles. For this purpose, 2 mL of the composition was applied directly to a defined area of the respective textile and left to act for 24 hours. Thereafter, a slight, light yellow residue could be seen on the white textile, whereas no residue at all was visible on the black textile here, after the application site had dried completely.

The white textile was subjected to a washing cycle with heavy-duty detergent at 40° C. for 80 minutes, the composition being completely washed out.

The black fabric was also subjected to washing at 40° C. for 80 minutes using a color detergent. The composition did not show any white spots or flakes on the black textile even after drying the application site and before washing, and was also residue-free after washing.

In summary, direct application of the composition to white and/or black cotton textiles does not result in any permanent stains, especially after washing the textiles.

The invention is not restricted to one of the embodiments described above, but rather may be modified in a variety of ways.

All of the features and advantages resulting from the claims and the description, including use characteristics and substance properties, may be essential to the invention both individually and in a wide variety of combinations.

1. An aluminum salt-free, antiperspirant and cosmetic and/or dermatological composition comprising at least one antiperspirant active ingredient, characterized in that the antiperspirant active ingredient is selected from the group of ionic liquids, wherein the at least one antiperspirant active ingredient has at least one cation and at least one anion, wherein the cation is selected from the group of: imidazolium, ammonium, phosphonium, guanidinium, pyrrolidinium, piperidinium and pyridinium, wherein the specified basic structures of the cations each have one or more substituents, of which at least one substituent is an alkyl substituent having a chain length of 1 to 10 carbon atoms.

2. The composition as claimed in claim 1, characterized in that the at least one cation is an alkylpyridinium cation.

3. The composition as claimed in either of the preceding claims, characterized in that the anion is selected from the group of halides.

4. The composition as claimed in any of the preceding claims, characterized in that at least one of the antiperspirant active ingredient comprises 1-hexylpyridinium chloride.

5. The composition as claimed in any of the preceding claims, characterized in that the at least one antiperspirant active ingredient is present to an extent of 1 to 25% by weight, particularly preferably 5 to 10% by weight, based on the total weight of the composition.

6. The composition as claimed in any of the preceding claims, characterized in that the composition has a pH of 3.5 to 7.0.

7. The use of an ionic liquid as antiperspirant active ingredient, characterized in that the ionic liquid has at least one cation and at least one anion, wherein the cation is selected from the group of: imidazolium, ammonium, phosphonium, guanidinium, pyrrolidinium, piperidinium and pyridinium, wherein the specified basic structures of the cations each have one or more substituents, of which at least one substituent is an alkyl substituent having a chain length of 1 to 10 carbon atoms.

8. The use as claimed in claim 7, characterized in that the ionic liquid comprises 1-hexylpyridinium chloride.

9. The use as claimed in claim 7 or 8, characterized in that the ionic liquid is used in combination with another antiperspirant active ingredient.

10. The use as claimed in claim 6 or 7, characterized in that the ionic liquid is used as the sole antiperspirant active ingredient in a cosmetic and/or dermatological preparation.
